(19) United States
(12) Patent Application Publication
(10) Pub. No.: US 2005/0163787 A1
(43) Pub. Date: Jul. 28, 2005

(54) Immunological adjuvant

(75) Inventors: Sveinbjorn Gizurarson, Reykjavik (IS); Sigriour Olafsdottir, Reykjavik (IS)

Correspondence Address: MERCHANT & GOULD PC, P.O. BOX 2903, MINNEAPOLIS, MN, 55402-0903, US

(73) Assignee: LYFJATHROUN HF, BIOPHARMACEUTICAL, Reykjavik (IS)

(21) Appl. No.: 10/486,592

(22) PCT Filed: Feb. 10, 2003
(86) PCT No.: PCT/IB03/00498

(30) Foreign Application Priority Data
Feb. 25, 2002 (IS) 6282

(51) Int. Cl. A61K 39/00

(52) U.S. Cl. 424/184.1

(57) ABSTRACT

A pharmaceutical composition to be administered to mammals via the mucosal route consisting of a vehicle system comprising mono-/dietherglycerides conjugated with a water soluble polymer groups selected from PEG's containing 2-30 polyoxyethylene units, an antigen and optionally a bacterial toxin for the augmentation of immune responses for vaccination, immunisation, treatment of allergy, treatment of cancer, treatment of infectious disease, treatment of an autoimmune disease, Alzheimer's, substance addiction or the treatment of a disease which is fully or partially controlled by the body immune system.

### FIELD OF THE INVENTION

The present invention relates to adjuvants and in particular to the use of mono-/dietherglycerides as adjuvants in a composition together with antigens for mucosal administration in humans or animals.

### BACKGROUND OF THE INVENTION

Parenteral administration (intramuscularly and subcutaneous) of antigens or vaccines are normally regarded as the most convenient way of administration. However, the injection presents a range of disadvantages. It requires the use of sterile syringes and may cause pains and irritations, particularly in the case of repeated injections, including the risk of infection. More significantly, intramuscularly injections are often poorly tolerated. There is often likely to be indurations (hardening of tissue), haemorrhage (bleeding) and/or necrosis (local death of tissue) at the injection site. Besides, untrained persons cannot administer injections.

Administration of attenuated virus, bacteria or parasite has been attempted intranasally as well as through other mucosal surfaces. The elicitation of an immune response through mucosal surfaces by antigens is not unexpected, because the modified live pathogen of the vaccine is following the natural route of infection of the wild-type pathogen inducing immunity through a sub-clinical infection. The use of modified live pathogen to induce immunisation is not without risks. However, using subunit vaccines, these more purified antigens are normally associated with poor responses and require therefore an effective formulation and an adjuvant to produce a clinically protective immune response.

Mucosal administration is currently receiving special interest, since this route is able to stimulate locally produced antibodies (secretory IgA antibodies) without the serious drawbacks associated with parenteral administration.

In order to be an attractive alternative to parenteral administration, mucosal administration via, for example, the intranasal route should be able to stimulate the local as well as the systemic immune system and the humoral as well as the cell-mediated immune system. Such immunizations need to be able to control these two systems, i.e. controlling factors such as cytokines, chemokines.

Upon mucosal, e.g. nasal, administration, the antigen must be applied to the mucosa in such a form that it is able to penetrate or be absorbed through the mucosa or the lymphoid issue to reach the immunocompetent cells. The antigen may also be administered on the nasal mucosa in such way that the antigen-detecting and antigen-presenting cells absorb the antigen and transport it to the local lymphoid system. In order to penetrate the mucus the vehicle must have a certain degree of biocompabbility with the mucus and hence have a certain degree of hydrophilicity and hydrophobicity.

Antigens are administered in the form of a suitable pharmaceutical composition. Depending on the chemical property of the antigen it may be necessary to consider different aspects in order to develop a composition for use in mammals.

The mucosal membrane contain numerous of dendritic and Langerhans cells, which are excellent antigen-detecting and antigen-presenting cells. The mucosal membranes are also connected to lymphoid organs, called mucosal associated lymphoid issue, which are able to forward an immune response to other mucosal areas. An example of such an epithelia is the nasal epithelial membrane, which consists of practically a single layer of epithelial cells (pseudostratifed epithelium) and the mucosal membrane in the upper respiratory tract is connected to the two lymphoid tissues, the adenoids and the tonsils. The extensive network of blood capillaries under the nasal mucosa and the high density of T and B cells, are particularly suited to provide a rapid recognition of the antigen and provide a quick immunological response. Other mucosal surfaces, which are attractive for the administration of antigens, are the nose, lungs, mouth, eye, ear, gastrointestinal tract, vagina, rectum, gills and the skin.

It has been described in the literature that the uptake of antigens from the nasal mucosa is dependent on the incorporation of absorption enhancing agents and/or adjuvants into the composition. If, for example, an adjuvant is not a part of the formulation it may result in that the antigen and/or the vaccine is absorbed and degraded, without recognition and, therefore, without stimulating an immunological response. Hence, there is always a need for an adjuvant system that is safe as well as effective.

### DESCRIPTION OF RELATED ART

U.S. Pat. No. 4,610,868 discloses a lipid matrix carrier for parenteral administration of drugs. This system requires a lipid matrix carrier comprising a hydrophobic compound, an amphipathic compound and a bioactive agent with a globular structure with 500-100000 nm in diameter. Here the hydrophobic compound may comprise a mixture of glycerides and the amphipathic compound may comprise a sphingolipid. Furthermore, this formulation may be administered into the nasal area. This system is not acceptable as a nasal formulation, due to the rapid clearance inside the nose and the large globular structure. Therefore, this system will be removed, into the stomach, by the cilia before the bioactive agent is released.

JP 309347/91 discloses an orally or nasally administered immunogen composition comprising an immunogen capable of immunising mammals using an adjuvant comprising of triglycerides with C6-26 residue of saturated or unsaturated fatty acid. Triglycerides have a reduced solubility compared to mono-/diglycerides having a hydrophilic group, moreover it not possible to attach an antigen to a triglyceride, which is possible with mono-/diglycerides.

In WO 94/17827 mono- and diglycerides are used for augmenting immune responses. These fatty glycerides are, however, not soluble in water and contain one or two hydrogen groups on the glyceride.

WO 99/02186 discloses a pharmaceutical composition comprising antigens and a adjuvant comprising ethoxylated mono and/or diglycerides for immunisation of humans. This is another example of fatty acid glycerides, which does not induce a immune response in the same manner as fatty alcohols. Furthermore, free fatty acids are slightly more irritative than fatty alcohols.

### OBJECTS OF THE INVENTION

This system used according to the invention relates to an antigen (including a vaccine) delivery system that provides a clear immunological response, in spite of the short contact-time with the mucosa, e.g. inside the nasal cavity, compared with the long duration following injectable vaccines.

The primary object of the invention is to provide a composition for mucosal administration or for the administration to the skin or even for the administration to the surface of plants, which is capable of control the systemic immune response (mainly of the IgG isotype) as well locally produced antibodies of the secretory IgA isotype at mucosal surfaces without causing unacceptable damage to the epithelial membrane. In plants the adjuvant is able to stimulate the plant immune defence system.

It is another object of the invention to provide a controlled delivery system for intranasal application, which are biocompatible with the mucus and which are capable of administering required amounts of antigens in small volumes.

It is also an object of the invention that this delivery system is usable for other mammalian surfaces such as the vagina, eye, mouth, lungs, ear, genital tract, gastrointestinal tract, rectum and the skin or in the fish to the gills.

In the present context, the term “an antigen” refers to a substance that causes the formation of an antibody or elicits a cellular response resulting in immunity of the host towards the pathogen.

The term “adjuvant” refers to a substance in a vaccine that is able to increase the body's immune response to an antigen present in the vaccine.

By “sequential administration” is meant that the adjuvant is administered alone followed by administration of the antigen, from immediately after up to 6 hours later or vice versa up to 30 minutes later.

### SUMMARY OF THE INVENTION

The present invention presents new and significantly improved adjuvant and a method for the administration of antigens, and therefore vaccines, using a new type of formulation. The method provides the possibility to control the immune response in the recipients of the antigen, both systemically and locally.

The objects of the invention are provided by using mono-/dietherglycerides with one or two C₆₋₂₄ residues of saturated or unsaturated alcohols and where the remaining group/groups are water soluble polymer groups selected from polyethyleneglycols (PEG's) containing 2-30 residues of polyoxyethylene or mixtures thereof as an adjuvant in a physiologically acceptable composition containing an antigen for mucosal administration in mammals for eliciting an immune response. The residues are more preferred saturated C₆₋₁₈ alcohol residues, most preferred C₈₋₁₂ alcohol residues. The water soluble polymers groups consist more preferably of PEG₂₋₃₀ residues of polyoxyethylene having 3-6 polyoxyethylene units. It is preferred that the PEG substituted glycerides, or the mixture thereof, has a concentration of from about 0.01% to about 30%, more preferred from about 0.5 to about 20%, most preferred from about 0,5 to about 5% by weight. It is preferred that the %-v/v ratio of substituted mono- and dietherglycerides is from about 0.1:99.9 to about 99.9:0.1, and more preferred from about 5:95 to about 95:5. It is preferred that the substituted etherglyceride, or the mixture thereof, has a concentration of from about 0.1% to about 99%, more preferred from about 0.5 to about 20%, most preferred from about 1 to about 15% by weight.

It is preferred that the antigen is a protein, drug or an enzyme, a vaccine against bacterial, viral, fungal, prion or parasitic infections, components produced by micro-organisms such as IgA-proteases, Protein p38, Protein p43 or mucinase cell component, drug or other substances where specific antigens are advantageous for the treatment or prophylactic treatment of diseases.

It is preferred that the composition further comprises a bacterial toxin or a derivative or subunit thereof, more preferred the bacterial toxin is cholera toxin or a derivative or subunit thereof, preferably cholera toxin B-subunit (CTB).

It is preferred that the antigen is in a particulate form or in a dissolved form. It is preferred that the composition further comprises one or more components selected from the group consisting of: surfactants, absorption promoters, water absorbing polymers, substances which inhibit enzymatic degradation, alcohols, organic solvents, oils, pH-controlling agents, solubilizers, stabilizers, HLB-controlling agents, viscosity controlling agents, preservatives, osmotic pressure controlling agents, propellants, air displacement, water, and mixtures thereof.

In a preferred aspect the adjuvants are used for the manufacture of a medicament for administration to humans. It is preferred that the administration is through a surface of the skin or a mucosal surface. It is preferred that the mucosal surface is selected from the group of mucosa surfaces of the nose, lungs, mouth, eye, ear, gastrointestinal tract, genital tract, vagina, rectum or gills.

According to the invention there is also provided a method of eliciting an immune response to an antigen in a mammal characterized by administering via the mucosal route one or more PEG-conjugated mono-/ditherglycerides as an adjuvant in a physiologically acceptable composition containing an antigen

According to the invention there is further provided a method of delivering an antigen to a mammal characterized by administering via the mucosal route one or more PEG-conjugated mono-/ditherglycerides as an adjuvant in a physiologically acceptable composition containing an antigen.

It is preferred that the antigen and the composition comprising the adjuvant are administered sequentially.

### A DETAILED DESCRIPTION OF THE INVENTION

It is often a cumbersome process to find a suitable adjuvant system. It has surprisingly been found that one or more mono-/dietherglycerides of formula (I): 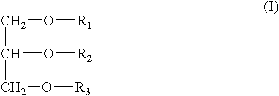
where R₁, R₂ and R₃ are either one or two C₆₋₂₄ residues of saturated or unsaturated alcohols and the remaining group/groups are water soluble polymer groups selected from PEG₂₋₃₀ residues of polyoxyethylene is useful as an adjuvant in a physiologically acceptable composition containing an antigen for mucosal administration in mammals for enhancing an immunological response

The invention also relates to a method for enhancing an immunological response characterized in administering to mammals via the mucosal route one or more mono-/dietherglycerides according to formula I as an adjuvant in a physiologically acceptable composition containing an antigen.

Examples of the chemical structure of the adjuvant used according to the invention is shown in (II)-(V) where the water soluble polymer group is a PEG₃₋₆ (PEG containing 3-6 residues of polyoxyethylene), i.e. —(OCH₂CH₂)₃₋₆—OH 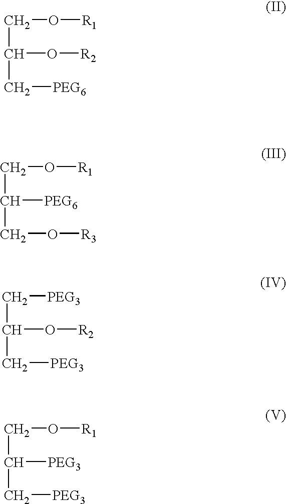
where R₁, R₂ and R₃ are C₆₋₂₄ residues of saturated or unsaturated alcohols, preferably saturated C₈₋₁₈ residues and more preferably saturated C₁₀₋₁₄ residues. In a preferred embodiment R₁ and R₂ are saturated C₆₋₁₈ alcohol residues and more preferably saturated C₈₋₁₂ alcohol residues.

The PEG₂₋₃₀ residues of polyoxyethylene, or derivatives thereof, have 2-30 polyoxyethylene units and more preferably 3-6 polyoxyethylene units.

The concentration of the glycerides exemplified by (II)-(V) in the composition may vary from 0.01% to 70%. The ratio between mono- and di-glycerides may vary from 0.1%:99.9% to 99.9%:0.1%, preferably from 5%:95% to 95%:5%. The chiral carbon in the glyceride may be in either S- or R-form.

The concentration of the mono-/dietherglycerides may be in the range of 0.01 to 70% wtw, preferably from 0.5 to 50% and more preferably from 1 to 30%.

The composition may further comprise a bacterial toxin or a derivative or subunit thereof. Preferably the toxin is cholera toxin or E. coli toxin and more preferably a subunit toxin from cholera, such as cholera toxin B subunit B (CTB). The addition of a toxin to the composition results in an improved yield of the formed antibodies.

The composition used according to the invention may be as a suspension, an emulsion or a dispersion providing the adjuvant in admixture with a dispersing or wetting agent, suspending agent, and/or one or more preservatives. Such compositions may also be suitable for use on mucosa such as the gastrointestinal, buccal, nasal, rectal, or vaginal mucosa, or for administration to plants and gills. Suitable dispersing or wetting agents are, for example, naturally occurring phosphatides, e.g., lecithin, or soybean lecithin; condensation products of ethylene oxide with e.g. a fatty acid, a long chain aliphatic alcohol, or a partial ester derived from fatty acids and a hexitol or a hexitol anhydride, for example polyoxyethylene stearate, polyoxyethylene sorbitol monooleate, polyoxyethylene sorbitan mono-oleate etc. Suitable suspending agents are, e.g., naturally occurring gums such as, e.g., gum acacia, xanthan gum, or gum tragacanth; celluloses such as, e.g., sodium carboxymethylcellulose, microcrystalline cellulose (e.g. Avicel® RC 591, methyl-cellulose; alginates such as, e.g., sodium alginate, etc. Suitable examples of preservatives for use in the compositions according to the invention are parabens, such as methyl or propyl p-hydroxybenzoate, and benzalkonium chloride.

For application to the rectal or vaginal mucosa suitable formulations for use according to the invention include suppositories (emulsion or suspension type), enemas, and rectal gelatin capsules (solutions or suspensions). Appropriate pharmaceutically acceptable suppository bases include cocoa butter, esterified fatty acids, glycerinated gelatin, and various water-soluble or dispersible bases like polyethylene glycols and polyoxyethylene sorbitan fatty acid esters. Various additives like, e.g., enhancers or surfactants may be incorporated.

For application to the nasal mucosa, nasal sprays and aerosols for inhalation are suitable compositions for use according to the invention. In a typically nasal composition, the active substance is present in the form of a particulate composition optionally dispersed in a suitable vehicle. The pharmaceutically acceptable vehicles and excipients and optionally other pharmaceutically acceptable materials present in the composition such as diluents, enhancers, flavouring agents, preservatives etc. are all selected in accordance with conventional pharmaceutical practice in a manner understood by the persons skilled in the art of formulating pharmaceuticals. After administration of a nasal composition according to the invention, the antigen may be adsorbed into the nasal mucosa. The adsorption to the mucosa is believed to lead to a less irritative effect than when e.g. a liquid vehicle e.g. containing a penetration enhancer or promoter is employed.

For application to the skin, the compositions according to the invention may contain conventionally non-toxic pharmaceutically acceptable carriers and excipients including microspheres and liposomes. The compositions include creams, ointments, lotions, liniments, gels, hydrogels, solutions, suspensions, sticks, sprays, pastes, plasters, and other kind of transdermal drug delivery systems. The pharmaceutically acceptable excipients may include emulsifying agents, anti-oxidants, buffering agents, preservatives, humectants, penetration enhancers, chelating agents, gelforming agents, ointment bases, perfumes, and skin protective agents.

Examples of emulsifying agents are naturally occurring gums, e.g. gum acacia or gum tragacanth, naturally occurring phosphatides, e.g. soybean lecithin, and sorbitan monooleate derivatives. Examples of antioxidants are butylated hydroxy anisole (BHA), ascorbic acid and derivatives thereof, tocopherol and derivatives thereof, butylated hydroxy anisole, and cysteine. Examples of preservatives are parabens, such as methyl or propyl p-hydroxybenzoate, and benzalkonium chloride. Examples of humectants are glycerin, propylene glycol, sorbitol, and urea. Examples of penetration enhancers are propylene glycol, DMSO, triethanolamine, N,N-dimethylacetamide, N,N-dimethylformamide, 2-pyrrolidone and derivatives thereof, tetrahydrofurfuryl alcohol, and Azone®. Examples of chelating agents are sodium EDTA, citric acid, and phosphoric acid. Examples of other excipients are edible oils like almond oil, castor oil, cacao butter, coconut oil, corn oil, cottonseed oil, linseed oil, olive oil, palm oil, peanut oil, poppyseed oil, rapeseed oil, sesame oil, soybean oil, sunflower oil, and teaseed oil; and of polymers such as carmelose, sodium carmelose, hydroxypropylmethylcellulose, hydroxyethylcellylose, hydroxypropylcellulose, chitosane, pectin, xanthan gum, carragenan, locust bean gum, acacia gum, gelatin, and alginates. Examples of ointment bases are beeswax, paraffin, cetyl palmitate, vegetable oils, sorbitan esters of fatty acids (Span), polyethylene glycols, and condensation products between sorbitan esters of fatty acids and ethylene oxide, e.g. polyoxyethylene sorbitan monooleate (Tween). The compositions mentioned above for topical administration may also be applied to the skin, the gills or to the outer surface of plants or they may be suitable for direct application or for introduction into relevant orifice(s) of the body, e.g. the rectal, urethral, vaginal or oral orifices. The composition may simply be applied directly on the part to be immunised such as, e.g., the mucosa.

Many mucosal compositions need some specialised mixture of excipients. Therefore many compositions may comprise one or more surfactants and/or absorption promoters and/or water absorbing polymers and/or substances which inhibit enzymatic degradation and/or alcohols, organic solvents, oils, pH-controlling agents, solubilizers, stabilisers, HLB-controlling agents, viscosity controlling agents, preservatives, osmotic pressure controlling agents, propellants, air displacement, water and mixtures thereof. The surfactants may be selected from nonoxynol, octoxynol, tweens, spans, sodium lauryl sulfate, sorbitan monopalmitate; absorbing promoters may be selected from polyoxyethylene alcohol ethers, bile salts and derivatives thereof, fusidic acid and derivatives thereof, oleic acid, lecitin, lysolechitins, Tweens 20-85, mono-/di- and triglycerides, chitosan, cyclodextriner; water absorbing polymers may be selected from glycofurols and derivatives thereof, polyethyleneglycol 200-7500 and derivatives thereof, polyvinylpyrrolidone, polyacrylic acid, propyleneglycol, gelatine, cellulose and derivatives thereof; substances which inhibit enzymatic degradation may be selected from aprotinin, DFP, carbopol; oils may be selected from vegetable oil, soybean oil, peanut oil, coconut oil, maize oil, olive oil, sunflower oil, Miglyols; pH-controlling agents may be selected from acetic acid, hydrochloric acid, nitric acid, potassium metaphosphate, potassium phosphate, sodium acetate, ammonia, sodium carbonate, sodium hydroxide, sodium borate, trolamine; solubilizers may be selected from alcohol, isopropyl alcohol, water, glycofurol, polyethyleneglycole 200-7500; stabilisers such as cyclodextrines; HLB controlling agents may be selected from Tween 20-85, Span 20-80, Brij 30-98, acacia; viscosity controlling agents may be selected from cellulose and derivatives thereof, Tweens and derivatives thereof, polyethyleneglycole and derivatives thereof, cetyl alcohol, glycerine, propylene glycol, sorbitol, gelatina; preservatives may be selected from benzalkonium salt, benzyl alcohol, phenol, thimerosal, pnenylmercuric nitrate, phenylethyl alcohol, chlorobutanol, cetylpyridinium chloride; osmotic pressure controlling agents may be selected from dextose, sodium chloride, mannitol; and propellants may be selected from dichlorodifluoromethane, dichlorotetrafluoroethane, trichloromonofluoromethane and other non-ozone damaging propellants such as butane; air displacement may be nitrogen.

The antigen employed in the composition used according to the invention is very suitable for inducing vaccination, immunisation, treatment of allergy, treatment of cancer, treatment of infectious disease, treatment of an autoimmune disease, Alzheimer's, addiction or the treatment of any disease which may be controlled by the body immune system. The antigen may be a protein, drug or an enzyme, a vaccine against bacterial, viral, fungal, prion or parasitic infections, components produced by micro-organisms such as IgA-proteases, Protein p38, Protein p43 or mucinase. The antigen may also be an allergen such as house dust mite, cat allergen, rye grass pollen, short ragweed pollen, midge, egg white, milk protein, bee venom, white faced hornet allergen etc. or components responsible for inducing autoimmune diseases such as myelin, insulin peptide B etc. The antigen may also be used as a vaccine for the treatment of infectious diseases, such as herpes, HIV, pappiloma, candida, multiple sclerosis, treatment of autoimmune diseases such as diabetes, hypo- and hyperthyroidism, psoriasis, arthritis, lupus, rheumatoid arthritis or the treatment of cancer.

The antigen may be used in a particulate form or a dissolved form. The composition is especially suitable to dissolve antigen; however, it is also easy to disperse a particulate form of the antigen in the composition. For vaccination and immunisation, the particulate form has often been the most advantageous form to use, but for inducing tolerance, e.g. for the treatment of allergy or autoimmune diseases, a dissolved antigen would be preferred.

The composition according to the invention is especially suitable for administration to humans, such as men, women, children, toddlers, adolescents, teenagers, adults and the elderly. The nature of the composition provides the possibility of influencing the immune response in various direction and therefore the composition may also be administered to subjects with various conditions such as splenectomized subjects, subjects with cancer, subjects using anticancer drugs, subjects using antiasthmatic drugs, subjects using antiinflammatoric drugs, subjects with hyper- and hypothyroidea. The composition is also useful in pregnant women and travellers.

The composition used according to the invention is also very suitable for administration to animals such as horse, sheep, dogs, cats, cow, pig, goat, rabbit, wild animals and laboratory animal such as mouse, rat, guinea pig, hamster, rabbit, dog, cat or monkey; to avian species such as chicken, turkey, duck or a wild bird; or to fish such as farm fishes e.g. salmon or aquarium fishes. For animals, the concentration of each component may need to be adjusted. For example for sheep, the nasal cavity has high humidity, which may require addition of water absorbing excipients to the composition and for fish, the formulation may need to be microencapsulated or in such a form that the formulation will be adsorbed to the gills.

The formulation may also be used to spray on plants, either on leaves, seeds, fruits, bark or wood such as the cucumber-plant, tomato-plant, potato-plant or the banana-plant

A composition may be administered to a variety of mucosal surfaces such as the mucosa of the nose, lungs, mouth, eye, ear, gastrointestinal tract, vagina, rectum or the gills, where especially the mucosa of the nose and mouth are very suitable for this formulation. In such cases the composition should be administered as nasal spray, nasal drops, nasal powder, nasal foam or as nasal ointment to the mucosal surface or the adenoids of the nose or as oral spray, oral drops, oral powder, oral foam or as oral ointment especially directed to the buccal area or to the tonsils of the mouth.

It is essential that the effective amount of the antigen can be administered in an appropriate volume. The volume should not exceed about 300 μl for a human subject. A larger volume can be disagreeable to the patient and will evidently drain out anteriorly through the nostrils or posteriorly toward the pharynx. The result is that a part of the antigen and/or the vaccine is lost form the absorption site. The volume is preferably from about 20 μl to about 150 μl and preferably administered into one nostril.

For the administration to the buccal and rectal area a volume not exceeding 10 ml should be used. For the administration to the eye and the ear a volume not exceeding 300 μl should be used. For the administration to the lungs, a volume not exceeding 2 ml should be used. For the administration to the vagina, a volume not exceeding 30 ml should be used. For the administration to the gastrointestinal tract a volume not exceeding 100 ml should be used.

The composition may also be used to deliver the antigen to the skin or to the surface of plants or to some specialised mucosal surfaces such as the gills.

### EXAMPLES

### Example I

30 Mice (BALB/c) are given intranasally 5 μl formulation, containing 1.5 μg cholera toxin B subunit in following formulations: (I) Isotonic saline; (II) 20% PEG-EG which is a mono-/diethylglyceride of C12 alcohol containing 6 polyoxyethylene (PEG₆) units, in isotonic saline; and compared with (III) 20% mixture of mono-/diglyceride of caprylic and capric acid (Imwitor®) solubilized with Tween 20 (22:78) in isotonic saline (from: WO 94/17827). Four weeks after the first vaccination, the mice received a booster containing the same vaccines. One week later, blood samples were withdrawn.

### Example II

Mice (BALB/c) are given intranasally 5 μl formulation, containing 1.5 μg tetanus toxoid in following formulations: (I) Isotonic saline; (II) 20%; (111) 10%; (IV) 5% and (V) 1% solution of PEG-EG which is a mono-/dietherglyceride of C12 alcohol containing 6 polyoxyethylene (PEG₆) units, in isotonic saline; Four weeks after the first vaccination, the mice received a booster containing the same vaccines. One week later, blood samples were withdrawn.

### Example III

Mice (BALB/c) are given intranasally 5 μl formulation containing 20% PEG-EG which is a mono-/dietherglyceride of C12 alcohol containing 6 polyoxyethylene (PEG₆) units in, isotonic saline where following antigens were added: (I) 1 μg HA influenza virus vaccine per mouse; (II) 1.5 μg D2 Herpes antigen; (III) 1.5 μg pertussis toxoid; (IV) 1 μg rgp120 HIV vaccine; (V) 1 μg IgA-protease as vaccine and (VI) 1 μg insulin peptide B. Four weeks after the first vaccination, the mice received a booster containing the same vaccines. One week later, blood samples were withdrawn and analysed.

### Example IV

Human volunteers are given intranasally 100 μl formulation, containing 12 μg influenza HA antigens in following formulations: (I) Isotonic saline; (II) 5% PEG-EG which is a mono-/dietherglyceride of C12 alcohol containing 6 polyoxyethylene (PEG₆) units, in isotonic saline; (III). Four weeks after the first vaccination, the participants received a booster containing the same vaccines. Weekly, during the study each volunteer is sampled (blood and saliva sample) for IgG and IgA analysis.

### Example V

Mice (BALB/c) are given 1.5 μg influenzae HA antigens in 5% PEG-EG which is a mono-/dietherglyceride of C12 alcohol containing 6 polyoxyethylene (PEG₆) units, in isotonic saline. The formulation is administered (I) 5 μl intranasally; (II) 5 μl orally into the mucosa of the mouth; (III) 5 μl vaginally; (IV) 5 μl rectally; and (V) 5 μl dermally. Four weeks after the first vaccinaton, the mice received a booster containing the same vaccines. One week later, blood samples were withdrawn.

1. A method for eliciting an immune response by mucosal administration in mammals which comprises administering to said mammals a physiologically acceptable composition comprising:
an adjuvant in the form of mono-/dietherglycerides of formula (I): 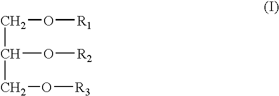
wherein either one or two of R₁, R₂ and R₃ are C₆₋₂₄ residues of saturated or unsaturated alcohols and the remaining group/groups are water soluble polymer groups selected from polyethyleneglycols (PEG's) containing 2-30 residues of polyoxyethylene or mixtures and an antigen.

2. The method according to claim 1, wherein one or two of R₁, R₂, and R₃ are selected from saturated C₆₋₁₈ alcohol residues.

3. The method according to claim 1, wherein the water soluble polymer groups consist of PEG₂₋₃₀ residues of polyoxyethylene having 2-30 polyoxyethylene units.

4. The method according to claim 1, wherein the PEG substituted glyceride, or the mixture thereof, has a concentration of from about 0.01% to about 30% by weight.

5. The method according to claim 1, wherein the %-v/v ratio of substituted mono- and dietherglycerides is from about 0.1:99.9 to about 99.9:0.1.

6. The method according to claim 1, wherein the substituted etherglyceride, or the mixture thereof, has a concentration of from about 0.1% to about 99% by weight.

7. The method according to claim 1, wherein chiral carbons in the etherglyceride are either S- or R-form, or mixtures thereof.

8. The method according to claim 1, wherein the antigen is a protein, drug or an enzyme, a vaccine against bacterial, viral, fungal, prion or parasitic infections, components produced by micro-organisms such as IgA-proteases, Protein p38, Protein p43 or mucinase.

9. The method according to claim 1, wherein the composition further comprises a bacterial toxin or a derivative or subunit thereof.

10. The method according to claim 9, wherein the bacterial toxin is cholera toxin or a derivative or subunit thereof, preferably cholera toxin B-subunit (CTB).

11. The method according to claim 1, wherein the antigen is a protein, cell component, drug or other substances where specific antigens are advantageous for the treatment or prophylactic treatment of diseases.

12. The method according to claim 1, wherein the antigen is in a particulate form.

13. The method according to claim 1, wherein the antigen is in a dissolved form.

14. The method according to claim 1, wherein the composition further comprises one or more components selected from the group consisting of: surfactants, absorption promoters, water absorbing polymers, substances which inhibit enzymatic degradation, alcohols, organic solvents, oils, pH-controlling agents, solubilizers, stabilizers, HLB-controlling agents, viscosity controlling agents, preservatives, osmotic pressure controlling agents, propellants, air displacement, water, and mixtures thereof.

15. The method according to claim 1 wherein the composition is immunogenic.

16. The method according to claim 1 wherein the composition is a vaccine.

17. The method according to claim 1, wherein the antigen is used for the treatment of an autoimmune disease, allergy, cancer, Alzheimer's or addiction.

18. The method according to claim 1 wherein the composition is administered to humans.

19. The method according to claim 18, wherein the administration is through a surface of the skin or a mucosal surface.

20. The method according to claim 19, wherein the mucosal surface is selected from the group of mucosa surfaces of the nose, lungs, mouth, eye, ear, gastrointestinal tract, genital tract, vagina, and rectum.

21-23. (canceled)

24. The method according to claim 1, wherein the glycerides have a structure selected from the group consisting of formulas (II), (III), (IV) and (V):

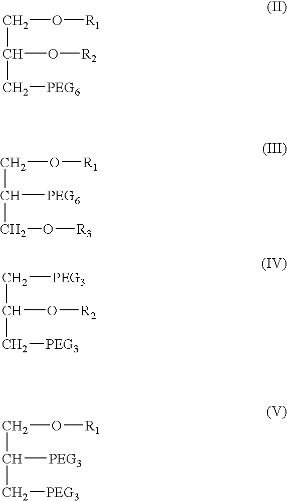

25. The method according to claim 1, wherein the antigen and the composition comprising the adjuvant are administered sequentially.

26. The method according to claim 1, wherein one or two of R₁, R₂, and R₃ are selected from saturated C₈₋₁₂ alcohol residues.

27. The method according to claim 1, wherein the water soluble polymer groups consist of PEG₂₋₃₀ residues of polyoxyethylene having 3-6 polyoxyethylene units.

28. The method according to claim 1, wherein the PEG substituted glyceride, or the mixture thereof, has a concentration of from 0.5 to about 20% by weight.

29. The method according to claim 1, wherein the PEG substituted glyceride, or the mixture thereof, has a concentration of from 0.5 to about 5% by weight.

30. The method according to claim 1, wherein the %-v/v ratio of substituted mono- and dietherglycerides is from 5:95 to about 95:5.

31. The method according to claim 1, wherein the substituted etherglyceride, or the mixture thereof, has a concentration of from 0.5 to about 20% by weight.

32. The method according to claim 1, wherein the substituted etherglyceride, or the mixture thereof, has a concentration of from 1 to about 15% by weight.
